(19) United States
(12) Patent Application Publication
(10) Pub. No.: US 2022/0062479 A1
(43) Pub. Date: Mar. 3, 2022

(54) Disinfection Bomb

(71) Applicant: Dennis Crook, Katy, TX (US)

(72) Inventor: Dennis Crook, Katy, TX (US)

(21) Appl. No.: 17/073,867

(22) Filed: Oct. 19, 2020

Related U.S. Application Data
(60) Provisional application No. 63/073,475, filed on Sep. 2, 2020.

(51) Int. Cl. A61L 2/22; A61L 2/26; A01N 31/02; A01N 31/08; A01N 25/06; A01N 33/12

(52) U.S. Cl. CPC A61L 2/22; A61L 2/26; A01N 31/02; A01N 31/08; A61L 2101/34; A01N 33/12; A61L 2202/25; A61L 2202/15; A01N 25/06

(57) ABSTRACT

This present invention relates to a portable disinfection device for dispersing a unique disinfecting solution into a room or enclosed space. The disinfecting solution may comprise one or more of a an ethyl or isopropyl alcohol, a lemon or fruit scent, an aldehyde, a chlorine compound, an oxidizing agent, a phenol, a quaternary ammonium, and a propellant, wherein the amount of the ethyl or isopropyl alcohol is at least 60% of the disinfecting solution by weight. The disinfecting solution is stored in a pressurized canister, and a release tab is pressed down for controlling the release of the disinfecting solution into the room from the canister. The disinfecting solution kills 99.9% of the germs and pathogens that it contacts.

### CROSS-REFERENCE TO RELATED APPLICATION

The present application claims priority to, and the benefit of, U.S. Provisional Application No. 63/073,475, which was filed on Sep. 2, 2020 and is incorporated herein by reference in its entirety.

### BACKGROUND

The present invention relates generally to the field of disinfecting devices. More specifically, the present invention relates to a disinfecting spray bomb device that disperses a disinfecting solution that has sanitizing agents for disinfecting airborne bacteria, viruses, germs and fungi within a confined or semi-confined area, such as a room in a home, office, school, hospital, restaurant, bar, i.e. plane, train, bus, and hotel room. The disinfecting spray bomb device is a spraying device similar to an insect fogger device, and offers complete area coverage for disinfecting a room of germs, bacteria, viruses, and the like. The disinfecting spray bomb device of the present invention is comprised of a pressure-activated canister that contains a novel disinfectant solution, a nozzle containing a disinfectant spray opening, and a release tab positioned on the top of the canister that, upon being pressed, releases the disinfectant solution throughout an entire room or other area to be treated. As the disinfectant solution leaves the nozzle spray opening, it is dispersed into the entire area of the room in one activation. The disinfectant solution of the present invention preferably comprises a mixture of alcohol and fragrance oil, and is capable of killing up to 99.9% of the germs, bacteria, virus, etc. within the treatment area. Accordingly, the present specification makes specific reference thereto. However, it could be appreciated that aspects of the present invention are also equally amenable to other like applications, devices and methods of manufacture.

Manual labor is currently required in order to maintain a properly sterilized environment within an enclosed space, and individuals oftentimes spend a considerable amount of time and/or effort cleaning and disinfecting a home, office, school, restaurant, business area, etc. Unfortunately, manually cleaning every surface in an enclosed space is time consuming, tiring, ineffective, and may result in some spots or areas being left unclean or untreated. Uncleaned or untreated surfaces can, in turn, lead to the spread of germs, bacteria, viruses and the like.

More specifically, manual cleaning and/or sanitizing suffers from a number of inherent drawbacks, such as missed areas and surfaces, re-contamination of a previously sanitized surface with a dirty sponge and/or mop, and the improper use and mixing of cleaning solutions. By way of example, individuals oftentimes come into contact with door knobs, handles, desks, tables, computer keyboards, telephones, tables, chair arms and other commonly utilized surfaces. An individual engaged in the cleaning or disinfecting of such surfaces must remember to clean the entirety of each of these surface, without cross-contaminating the same with a dirty rag, mop or other cleaning device that previously came into contact with a contaminated surface. This in turn requires the frequent cleaning or replacement of the tools being used during the cleaning operation, which further adds to the time and expense of the cleaning operation. Additionally, cleaning of the various surfaces does nothing to cleanse the air in an enclosed space of harmful airborne, microbes, germs, pathogens and the like.

Further, in today's environment and the ongoing COVID-19 pandemic, the demand for sterilized environments is of utmost concern and importance. Therefore, members of a cleaning staff must regularly sterilize environments to ensure the areas visited by the public, customers, patients and the like are clean and free of harmful pathogens. The sterilization process is oftentimes accomplished through the use of chemicals sprays and/or wipes that have been saturated or treated with a disinfectant or other chemical to help sterilize the area. Unfortunately, this sterilization and cleaning effort is also labor intensive, and may still result in some surfaces in the area of concern remaining untreated given the fact that it is not possible for the cleaning staff to visibly see which surfaces have been cleaned, and which have not.

Therefore, there exists a long felt need in the art for a portable or personal disinfection bomb product that can be employed within homes, businesses, restaurants, schools, hospitals and other enclosed spaces to effectively disinfect the same and eliminate unpleasant odors, bacteria, germs, viruses, and the like. There is also a long felt need in the art for a portable disinfection bomb product containing a novel combination of disinfecting solution and fragrance oils that provides immediate disinfection and a pleasant fragrance. Moreover, there is a long felt need in the art for a disinfection bomb product that eliminates the manual labor and long hours needed to provide an effective cleaning, and that ensures that all areas are properly cleaned and/or disinfected. Additionally, there is a long felt need in the art for a portable disinfecting product that requires minimal effort on the part of the user, and that effectively treats the air in the enclosed room as well as all of the various surfaces. Finally, there is a long felt need in the art for a portable disinfection bomb product that kills up to 99.9% of the germs, bacteria and other harmful microbes in the treated area, is relatively inexpensive to manufacture, and that is both safe and easy to use.

The subject matter disclosed and claimed herein, in one embodiment thereof, comprises a disinfecting spray product for killing germs, viruses and bacteria in an enclosed area. The product comprises a canister having a predetermined shape and size, and that is formed of a predetermined material. The canister contains a pressurized disinfecting solution therein that is designed to kill 99.9% of germs, bacteria and the like on contact, and that leaves a pleasant fragrance in the air. A release tab is disposed on a top portion of the canister to enable a user to release the disinfecting solution into the enclosed space in the form of a fog, mist or spray, thereby assuring 100% coverage.

The disinfecting solution of the present invention may have a composition comprised of essentially natural ingredients. In a preferred embodiment, the essential components of the disinfecting solution are ethanol and one or more fragrant oils. More specifically, ethyl alcohol (ethanol) or isopropyl alcohol are used as anti-microbial agents within the disinfecting solution. Ethanol is considered an effective substance against a large spectrum of microorganisms which can linger on the skin, and also evaporates rapidly once released from the container, thereby ensuring a relatively quick drying time so that the enclosed space can quickly be returned to its intended and productive use.

In this manner, the disinfecting bomb spray product of the present invention accomplishes all of the forgoing objectives, and provides a relatively safe, easy, convenient and cost-effective solution to the problem of having to completely sanitize an enclosed space in a relatively short period of time and with a limited labor force. The disinfection bomb spray product of the present invention is also user friendly inasmuch as it is less expensive than traditional manual cleaning operations, and requires minimal manual labor to accomplish. Finally, the solution used in the disinfecting product effectively kills 99.9% or all germs, bacteria, pathogens and the like that it comes into contact with, thereby resulting in a disinfected and fragrant environment in much less time than other cleaning processes.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

By way of background, aerosols are made up of tiny liquid droplets which get sprayed or otherwise propelled into the air, and are primarily dispersed from a pressurized canister. Historically, aerosol technology has been used in painting and pest control applications, as well as in personal care products, such as hairsprays, deodorants, inhalers, and the like. However, disinfection via a fogger with a suitable disinfectant represents a very special technology. More specifically, the aerosol disinfectant is atomized into tiny droplets using an aerosol disinfectant fogger, carried along by the air current (e.g., via an HVAC system) and dispersed into the ambient air in a quick and efficient manner. Further, the air current evenly distributes the tiny droplets of disinfecting solution throughout the entire room, which provides a significant advantage over other conventional methods of cleaning, which typically require significant manual effort and time, and oftentimes results in surfaces being missed.

The subject matter disclosed and claimed herein, in one embodiment thereof, discloses a disinfecting spray bomb device for sanitizing the air and exposed surfaces in a room or an enclosed space or area. The device comprises a canister storing a pressurized quantity of disinfecting solution that is comprised of an alcohol, a fragrance oil and a propellant. The container has a release tab that may be depressed by a user to activate the canister and cause the emission of an aerosol spray of the disinfecting solution upwardly to spread throughout the room and coat exposed surfaces with a thin, quick-drying film.

In a further embodiment of the present invention, a disinfecting solution for sanitizing the exposed surfaces in a room or an enclosed space is disclosed and is comprised of one or more of an ethyl or isopropyl alcohol, a lemon or fruit scent, an aldehyde, a chlorine compound, an oxidizing agent, a phenol, a quaternary ammonium, and a propellant. The amount of the ethyl or isopropyl alcohol is at least 60% of the disinfecting solution by weight. In a further embodiment of the invention, ethanol (i.e., ethyl alcohol) may comprise approximately 75-90% of the disinfecting solution by weight. In said embodiment, the ethanol can be mixed with water to slow the drying time of the disinfecting solution of the present invention, thus, creating a longer contact time with the unsanitary surface or surfaces. By way of background, ethanol typically requires a contact time of at least 10 seconds to kill Staphylococcus aureus and Streptococcus pyogenes. Also, it has been shown that a 10 second contact time allows the ethanol to kill microbes such as Pseudomonas aeruginosa, E. coli, Pseudomonas aeruginosa, etc.

In a further embodiment of the present invention, a method of sanitizing a room or enclosed space is disclosed and comprises the steps of initially providing a disinfecting bomb spray device comprised of a pressurized canister of disinfecting solution, and placing the canister at a position near the center of the room. The canister is activated by pressing down on a release tab present at the top of the canister. After activation, the room may be closed, and the disinfecting solution is dispensed throughout the room in the form of an aerosol spray to sanitize the room and all of the surfaces therein. Once the disinfecting process is completed and the solution from the canister consumed, the room is reopened and can return to productive use.

In a further embodiment of the present invention, a disinfecting spray device is provided and includes a canister that contains a pressurized disinfecting solution and a propellant. The canister further comprises a release tab and a nozzle fitted on top of the canister. The nozzle is in fluid communication with the disinfecting solution stored inside the canister so that when the release tab is depressed the pressurized disinfecting solution is dispensed through the nozzle opening and into the enclosed area.

In yet another embodiment of the present invention, an aerosol disinfectant dispersing device for releasing an aerosolized disinfecting solution to sanitize a room is provided and includes a canister having an aerosol disinfecting solution composed of alcohol and fragrance oil and a sensor or a timer. The contents of the canister are contained under pressure, and the canister has a top end having an opening that extends into an interior of the canister. A nozzle is coupled to the canister and is in fluid communication with an interior of the canister to disperse the disinfecting solution in accordance with instructions received by the sensor or the timer. The disinfecting solution is dispersed into the room through a nozzle opening.

The aerosol spray disinfecting bomb of the present invention generates a relatively fine mist or fog of disinfecting solution present in the pressurized canister that, when dispersed, enables whole rooms or other selected areas to be effectively treated in a limited amount of times. One benefit of using the relatively fine mist or fog produced by the disinfecting bomb device of the present invention is that it attacks bacterial, viruses, germs and the like both in the air and on a surface. Further, the relatively fine droplets can also access hard-to-reach areas, thereby providing a more thorough cleaning and disinfecting.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the disinfecting spray bomb product of the present invention in accordance with the disclosed architecture;

FIG. 2 illustrates a diagrammatic representation of one potential formulation for the disinfecting solution of the present invention in accordance with the disclosed architecture;

FIG. 3 illustrates a perspective view of one potential embodiment of the nozzle portion of the portable disinfectant spray bomb product of the present invention in a non-activated state in accordance with the disclosed architecture;

FIG. 4 illustrates a perspective view of one potential embodiment of the nozzle portion of the portable disinfectant spray bomb product of the present invention in an activated state in accordance with the disclosed architecture; and

FIG. 5 illustrates a perspective view of one potential embodiment of the portable disinfectant spray bomb product of the present invention in an activated state and being used to disinfect an entire room in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a portable disinfection bomb product that can be employed within a home, business, restaurant, school, hospital or other enclosed space to effectively disinfect the same, and eliminate unpleasant odors, bacteria, germs, viruses and the like. There is also a long felt need in the art for a portable disinfection bomb product containing a novel combination of disinfecting solution and fragrance oils that provides immediate disinfection and leaves a pleasant fragrance for subsequent users of the enclosed space. Moreover, there is a long felt need in the art for a disinfection bomb product that eliminates the manual labor and long hours typically associated with manual cleaning processes, and that ensures that all areas are properly cleaned and/or disinfected. Additionally, there is a long felt need in the art for a portable disinfecting product that requires minimal effort on the part of the user, and that effectively treats the air in the enclosed room as well as all of the various surfaces. Finally, there is a long felt need in the art for a portable disinfection bomb product that kills up to 99.9% of the germs, bacteria and other harmful microbes in the treated area, is relatively inexpensive to manufacture, and that is both safe and easy to use.

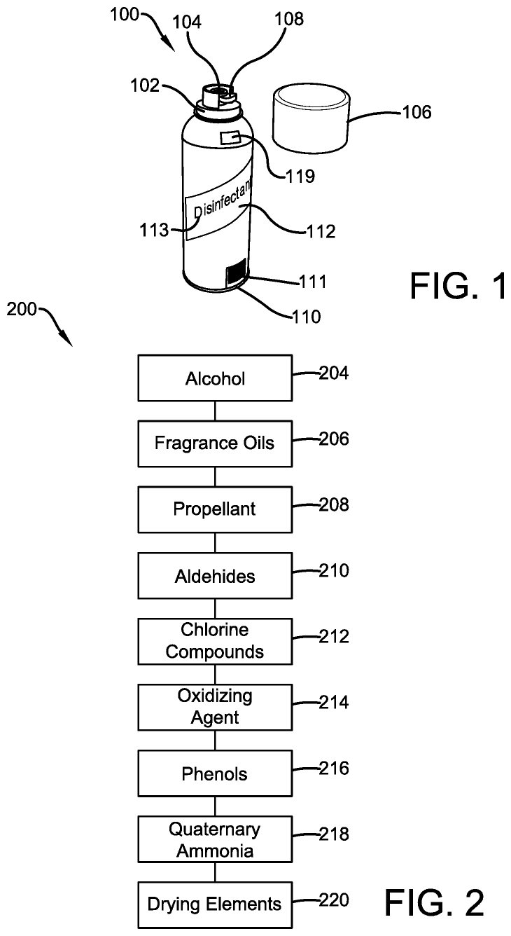

Referring initially to the drawings, FIG. 1 illustrates a perspective view of one potential embodiment of the disinfecting spray bomb product 100 of the present invention in accordance with the disclosed architecture. The disinfecting spray bomb product 100 is used for killing germs, viruses, bacteria and other pathogens in an enclosed area such as a room or an office. The disinfecting bomb 100 comprises a pressurized canister 112, a nozzle or other opening 104, a release tab 108, and a disinfecting solution 200 stored in the pressurized canister 112 and in fluid communication with the nozzle 104 when the release tab 108 is depressed or otherwise in an open condition.

The pressurized canister 112 may be of a predetermined shape, size and material, such as metal or plastic. The pressure in the canister 112 is preferably between 140 and 160 psig, and should not exceed 160 psig for plastic canisters or 200 psig for metal containers. The canister 112 is comprised of a base 110 on which the spray bomb 100 is placed upright, and the canister 112 stores sanitizing and disinfecting agents such as alcohol and fragrance oils along with a propellant to disperse them into an enclosed room. More specifically, a release tab 108 is present on the top portion 102 of the canister 112, which may be activated by pressing down thereon to release the disinfecting solution 200 into the room through a nozzle spray opening 104 in a 360-degree direction. The top portion 102 of the canister 112 is covered with a lid 106 to prevent accidental pressing of the release tab 108, as well as to cover the spent canister nozzle for disposal.

The disinfecting solution 200 contained within the canister 112 is under pressure and comprises a propellant to assist with the dispensing. Once activated, the spray bomb 100 disperses the disinfecting solution 200 in a manner similar to a conventional aerosol spray can from the nozzle spray opening 104. For this reason, some of the structure relating to conventional aerosol sprays is not shown or described in greater detail

FIG. 2 illustrates a diagrammatic representation of one potential formulation for the disinfecting solution 200 of the present invention in accordance with the disclosed architecture, which is generally comprised of an alcohol 204, a fragrant oil 206 and a propellant 208. More specifically, the alcohol 204 is preferably an ethyl or isopropyl alcohol that makes up at least 60% of the disinfecting solution 200 by weight. In a further embodiment of the invention, the ethanol (i.e., ethyl alcohol) content may comprise approximately 75-90% of the disinfecting solution by weight. Ethanol is a preferred co-solvent and enhances the solubility of the solution 200 and evaporates relatively quickly after release. More specifically, the volume of ethanol in the solution contributes to the rate at which the disinfectant solution 200 evaporates, thereby leaving behind the active component of the disinfectant which will continue to have a beneficial effect on any of the surfaces it contacts. Nonetheless, the ethanol may also be mixed with water to slow the drying time of the disinfecting solution 200, thereby creating a longer contact time with the unsanitary surfaces in which it contacts.

As stated above, the solution 200 preferably also comprises a fragrant oil 206, such as a lemon, citrus, lavender, pine, cinnamon or any other scent that may be appealing to the user. For example, fragrance oil ingredients such as acetyl cedrene (a member of the fragrance structural group alkyl cyclic ketones) to impart wood scent like ambergris, dihydromyrcenol (monoterpenoid) to impart a powerful, fresh lime-like, citrusy-floral and sweet odor with little or no terpenic undertones, linalyl acetate to impart lavender fragrance, or dipropylene glycol to impart slightly ethereal and sweet fragrance may be used in the disinfecting solution 200. The fragrant oil preferably comprises between 0.5 and 5% of disinfecting solution 200 by weight.

The propellant 208 is used to substantially discharge all of the disinfecting solution 200 from the canister 112, and may be any propellant generally known in the art provided that the same does not chemically react with the other ingredients of the disinfesting solution 200, such as a form of butane, pentane or propane.

Additionally, the disinfecting solution 200 may further comprise one or more of an aldehyde 210, chlorine compound 212, oxidizing agent 214m phenol 216 and/or quaternary ammonium 218. The aldehyde 210, if present, is used to eliminate bacteria and other parasites, as well as to prevent the growth of such organisms and may be any of a formaldehyde, paraformaldehyde, glutaraldehyde, and/or ortho-phthalaldehyde (OPA). The aldehyde 210 preferably comprises between 1 and 5% of the disinfecting solution 200, by weight.

The chlorine compounds 212, if present, is used as a sterilizing agent, and may be comprised of any of a hypochlorite, chlorine, dioxide, calcium hypochlorite, sodium dichloroisocyanurate, sodium dichloro-striazinetrione, and/or sodium hypochlorite. The chlorine compounds 212 preferably comprise between 0.5 and 5% of the disinfecting solution 200, by weight. Similarly, the oxidizing agents 214, if present, are used as disinfecting agents, and may be any of a hydrogen peroxide, a peroxyacetic acid, or the like. The chlorine compounds 212 preferably comprise between 1 and 10% of the disinfecting solution 200, by weight.

The phenols 216, if present, are used as binding agents, such as with the fragrance oils 206, and may be comprised of any of a hexachlorophene, thymol or the like. The phenols 216 preferably comprise less than 1% of the disinfecting solution 200, by weight. Similarly, the quaternary ammonium compounds 218, if present, are also used as disinfecting and/or sterilizing agents and may be comprised of an alky dimethyl benzyl ammonium chloride or the like. The quaternary ammonium compounds 218 preferably comprise between 0.5 and 5% percent of the disinfecting solution 200, by weight.

In a further preferred embodiment of the present invention, the disinfecting solution 200 may also comprise one or more drying elements 220 including, but not limited to, a calcium sulfate, sodium sulfate, calcium chloride and/or magnesium sulfate which can help speed the drying of the disinfecting solution 200 to more efficiently return the enclosed room to productive use. The drying elements 220, if present, preferably comprise between 0.5 and 15%, and more preferably between 2 and 7%, of the disinfecting solution 200, by weight.

In a further embodiment, the disinfecting solution 200 may further comprise additional ingredients that support the performance or prolong the effectiveness of the disinfecting solution 200. For example, a pH adjuster may be used to control the pH level of the disinfecting solution 200 to ensure stability and maximize performance. Also, as previously mentioned, water may also be used as a diluent to adjust the concentration of ingredients in the solution 200 to, for example, deliver target benefits.

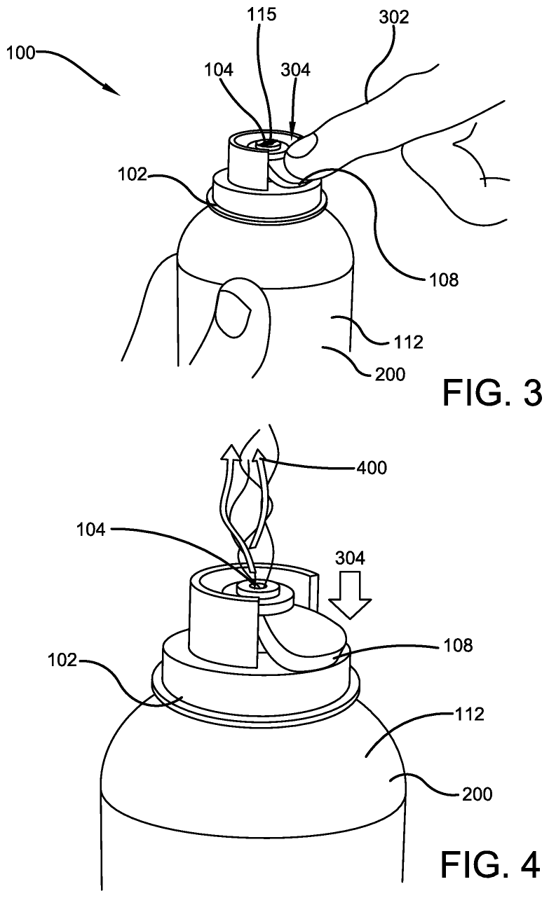

FIG. 3 illustrates a perspective view of one potential embodiment of the nozzle portion 104 of the portable disinfectant spray bomb product 100 of the present invention in a non-activated state in accordance with the disclosed architecture. More specifically, to release the disinfecting solution 200 stored in the pressurized canister 112 into the room or other enclosed space, a user 302 presses down 304 on the release tab 108 with relatively minimal force to activate the nozzle 104 and release the disinfecting solution 200 from the canister 112. More specifically, the nozzle 104 is in fluid communication with an interior of the canister 112 when the release tab 108 is pressed down 304, thereby facilitating the aerosol and the disinfecting solution 200 to be released from the canister 112 and into the room. In this manner, the disinfecting solution 200 is distributed around the room evenly to disinfect the entire room, as well as the air contained therein.

The nozzle 104 may also be provided with a flow diverter 115 which can direct the spray in a particular direction. For example, if the user places the canister 112 against the wall, the flow diverter can direct the spray into the room and away from the wall, so that the wall does not become overly wet with the solution 200. The nozzle may also include a screen or mesh to control the droplet size, with the droplet size ranging from about 5 microns to about 50 microns and having a predefined dispensing rate and an effective solution concentration of between 10 to 90%. Droplets sizes for use in the present invention range between 5-50 microns (μm) in diameter have proven to be most effective. Preferably, the droplet size is between 10 and 35 microns. Droplets of this size are ideal to tackle pathogens, vector carriers and pests. In addition, the effective portions of formulations can be applied in concentrations ranging from 10-90%, with the remaining portions making up propellant, stabilizers, drying agents or other non-treatment components and more preferably from 30-80% and at flow rates of up to 0.52 quarts per minute (31.7 quarts per, hour or nearly 8 gallons an hour). Application of disinfectants, sanitizing solutions and biocides via aerosol or fogging can significantly reduce the number of viable infectious pathogens in a particular area. Foggers produce micro droplets that float in the air for approximately 10 minutes after application, reaching the most inaccessible parts of a room or area where conventional cleaning or spraying typically can't reach. The smaller the droplet, the longer the particles will remain airborne. Approximately 1 ounce of disinfectant solution 200 will typically cover around 1,000 cubic feet.

FIG. 4 illustrates a perspective view of one potential embodiment of the nozzle portion 104 of the portable disinfectant spray bomb product 100 of the present invention in an activated state in accordance with the disclosed architecture. In use, to release the disinfectant solution 200 comprehensively and evenly throughout a room, when the release tab 108 is pressed in a downward direction 304 towards the canister 112, the nozzle 104 releases the disinfecting spray 400. After use or after a release of a pre-determined amount of solution 200, the release tab 108 is manually or automatically released, thereby allowing the released disinfectant solution 200 to escape as a fog 400 in a very controlled way in 360-degree direction.

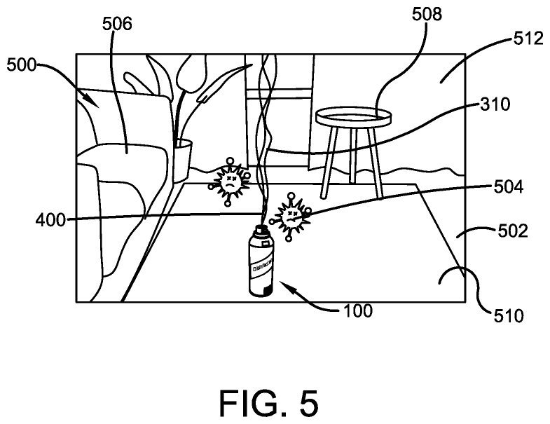

FIG. 5 illustrates a perspective view of one potential embodiment of the portable disinfectant spray bomb product 100 of the present invention in an activated state and being used to disinfect an entire room 500 in accordance with the disclosed architecture. More specifically, the disinfecting spray bomb 100 is being used in a room 500 comprising a floor 502, a sofa 506, a stool 508, carpet 510 and walls 512. The disinfecting spray bomb 100 is placed appropriately for use with an open lid, in a location that is substantially near the center of the room 500, and at a height calculated to allow the spray 400 that is ejected to be able to fall onto or contact all the surfaces in the room 500 including those mentioned above. More specifically, the spray 400 is propelled outwardly and upwardly to a height near or at the ceiling of the room 500. The spray droplets 400 then fall downward and impinge on succeeding upwardly propelled droplets. The net effect is a mushrooming of the spray droplets 400 throughout the entire room, descending to contact all exposed surfaces. The spray 400 kills 99.9% of the germs and pathogens 504 present in the room 500 to provide a sanitized environment. To completely sanitize and disinfect the room 500, the room should remain closed for 1-2 hours after application.

As previously stated, the disinfecting bomb spray 100 according to the present invention kills up to 99.9 percent of all germs 504 existing in the air space and all the surfaces where the disinfecting solution 200 is emitted. The disinfecting solution 200 may be a room temperature (e.g., 20° C. to 35° C.) substance that can be dispersed as a fog during operation. In other embodiments, the disinfecting solution 200 may be used at temperatures in the range of between about −40° C. to 100° C.

The disinfecting bomb spray 100 of the present invention may also include other features in combination to increase the utility and value to the spray product 100 when sold commercially. In a preferred embodiment, the spray bomb 100 may have a metered valve so that upon pressing of the release tab 108, only a pre-determined amount of disinfecting solution 200 is released into the room 500. Likewise, the disinfecting bomb spray 100 of the invention can be appropriately decorated to fit into any room decor. The disinfecting bomb spray 100 may have a barcode 111 or a QR code which can be scanned using a smartphone camera to order new spray 100 products from a retailer and/or manufacturer.

The canister may also include a sensor or timer 119 for use in activation. For example, the sensor module or timer 119 may include one or more status sensors, such as motion, heat sources, and/or other sensors that prevent the device 100 from initiating while the sensors detect activity in the room. Further, a timing controller can be programmed with a specific time duration as desired by the user to automatically release the disinfecting solution 200 into the surroundings. If the sensor module senses conditions that are not suitable or ideal for disinfection of the room, the defogger 100 may not release the disinfecting solution 200.

In an alternate embodiment of the present invention, a sensor module may be present that may include one or more status sensors, such as motion, heat sources, and/or other sensors, that prevents the bomb device 100 from initiating or activating when, for example, the sensors detect activity in the room 500. Further, a timing controller can be programmed with a specific time duration as desired by the user to automatic release the disinfecting solution 200 into the surroundings. If the sensor module senses conditions that are not suitable or ideal for disinfection of the room, the bomb spray 100 may not release the disinfectant spray 400 into the surroundings.

The disinfection spray bomb 100 of the present invention is useful to disinfect and sanitize all type of surfaces including, without limitation, fabric, canvas, leather, PVC, glass, vinyl, plastic, rubber, metal, wood, gel coat, fiberglass, etc., and can be used in homes, offices, clinics, laboratories, nursing homes and the like. The spray bomb 100 provides effective disinfection of all surfaces frequently touched by individuals in an enclosed space, and is effective against all types of microorganisms including, without limitation, SARS-CoV-2, H1N1 influenza virus, HIV-1 virus, Types 1&2 viruses, cold and flu viruses, and other similar pathogens.

The disinfection spray bomb 100 of the present invention provides whole room disinfection, and helps to eliminate some of the errors associated with prior art cleaning methods and reduce the time required to disinfect an enclosed area. The disinfection spray bomb 100 of the present invention also limits the use of chemicals and bleaching agents, which benefits the environment as well as the housekeeping employees who would otherwise be in frequent contact with the same. The spray bomb 100 disinfects the air and the exposed surfaces, and also helps in infection control (i.e., prophylaxis).

The canister 112 may be available in different sizes and colors to accommodate a user's needs and/or preferences. For example, a canister may include enough disinfecting solution 200 to treat areas up to 6,000 cubic feet, or more. Nonetheless, the exact size, measurement, construction and design specifications of the unique bomb spray 100 of the present invention may vary upon manufacturing, or the particular materials that are being is used, as well as the size of the enclosed area to be treated. For example, depending on the room size, more than one bomb spray device 100 may be required to substantially sanitize the area. The disinfecting spray bomb 100 may be commercially available as a single pack of spray bombs of the same or different fragrance. Alternatively, the disinfecting spray bomb 100 may be commercially available as a single unit. Additionally, the canister 112 may have a name tag, name badge, laser-graving, customizable colors and fonts, embroidery and/or prints 113.

Use of the portable disinfecting fogger product 100 is very simple and straight forward. Another productive way to use portable disinfecting spray bomb product 100 is to insert it in an optional wall mounted housing unit. The housing unit can be fabricated of plastic or stainless steel, and the mounting brackets may be manufactured of a metal alloy.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “disinfecting spray bomb”, “disinfection bomb”, “disinfecting fogger”, “bomb spray”, and “disinfecting fogger product” are interchangeable and refer to the portable disinfecting spray bomb 100 of the present invention.

Notwithstanding the forgoing, the portable disinfecting spray bomb 100 of the present invention and its various components can be of any suitable size and configuration as is known in the art without affecting the overall concept of the invention, provided that it accomplishes the above stated objectives. One of ordinary skill in the art will appreciate that the size, configuration and material of the portable disinfecting spray bomb 100 and its various components as shown in the FIGS. are for illustrative purposes only, and that many other sizes and shapes of the portable disinfecting spray bomb 100 and its components are well within the scope of the present disclosure. Although the dimensions of the portable disinfecting spray bomb 100 and its components are important design parameters for user convenience, the portable disinfecting spray bomb 100 and its components may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A disinfecting device comprising:
a container sized and configured to receive a disinfecting solution under pressure, the container having a flat base on one end and a nozzle on a second end opposite the flat base, wherein the nozzle is movable between an open and a closed position, and is substantially surrounded by a collar; and
wherein the disinfecting solution is contained within the container and held at a pressure that is higher than an ambient pressure, and further wherein the disinfecting solution is comprised of an alcohol, a fragrance, a sanitizing agent, and an oxidizing agent.

2. The disinfecting device as recited in claim 1, wherein the nozzle is comprised of a flow diverter to direct the disinfecting solution in a particular direction as it is discharged from the container.

3. The disinfecting device as recited in claim 1, wherein the alcohol is one of an ethyl alcohol or an isopropyl alcohol and ranges between 60% and 90% of the disinfecting solution by weight.

4. The disinfecting device as recited in claim 1, wherein the oxidizing agent is one of a hydrogen peroxide and a peroxyacetic acid and ranges between 1 and 10% of the disinfecting solution by weight.

5. The disinfecting device as recited in claim 1, wherein the sanitizing agent is one of a chlorine compound and a quaternary ammonium compound and ranges between 0.5% and 10% of the disinfecting solution by weight.

6. The disinfecting device as recited in claim 1, wherein the disinfecting solution further comprises a propellant that produces a droplet size of between 5 and 50 microns when released from the container.

7. The disinfecting device as recited in claim 1, wherein the disinfecting solution further comprises a propellant that produces a flow rate of up to 0.52 quarts per minute.

8. The disinfecting device as recited in claim 1, wherein the disinfecting solution is further comprised of one of a hexachlorophene and a thymol.

9. The disinfecting device as recited in claim 1, wherein the disinfecting solution further comprises at least one drying element selected from a group consisting of a calcium sulfate, a sodium sulfate, a calcium chloride and a magnesium sulfate, and further wherein the at least one drying element may range from between 0.5 to about 15% of the disinfecting solution by weight.

10. A sanitizing fogger comprising:
a container for holding a content under a pressure that is greater than an ambient pressure, wherein the container comprises a flat bottom, a nozzle, and one of a sensor or a timer for use in activation;
the nozzle being repositionable between an open position and a closed position, and the container including a cap for securing the nozzle; and
a disinfecting solution stored in the container at the pressure and comprising an ethyl alcohol or an isopropyl alcohol in an amount ranging from 60% to 90% of the disinfecting solution by weight.

11. The sanitizing fogger as recited in claim 10, wherein the disinfecting solution comprises a propellant that with the pressure, produces a disinfecting solution particle droplet size ranging from 10 to 35 microns.

12. The sanitizing fogger as recited in claim 10, wherein the disinfecting solution further comprises a fragrance, a sanitizing agent, and an oxidizing agent.

13. The sanitizing fogger as recited in claim 10, wherein the disinfecting solution further comprises at least one drying element selected from a group consisting of a calcium sulfate, a sodium sulfate, a calcium chloride and a magnesium sulfate, wherein the at least one drying element comprises between 0.5 to about 15% of the disinfecting solution by weight.

14. The sanitizing fogger as recited in claim 10, wherein the disinfecting solution comprises at least one of an aldehyde, a chlorine oxidizing agent, a phenol, and a quaternary ammonium compound.

15. The sanitizing fogger as recited in claim 10 and having a disinfecting solution dispersal flow rate of up to 0.52 quarts per minute.

16. A method of using a disinfecting device comprising the steps of;
selecting an area to be disinfected;
providing a canister having a disinfecting solution, wherein the canister is configured to hold the disinfecting solution under a pressure that is greater than an ambient pressure, and further wherein the canister is comprised of a bottom and a nozzle having an open position and a closed position;
filling the canister with the disinfecting solution at the pressure;
repositioning the nozzle from the closed position to the open position; and
dispersing the disinfecting solution from the canister, wherein the nozzle produces a disinfecting solution droplet size of between 5 and 50 microns

17. The method of using a disinfecting device as recited in claim 16, wherein the disinfecting solution is comprised of one of an ethyl alcohol or an isopropyl alcohol ranging between 60% and 90% of the disinfecting solution by weight.

18. The method of using a disinfecting device as recited in claim 17, wherein the disinfecting solution further comprises at least one of a fragrance, an aldehyde, a chlorine oxidizing agent, a phenol, and a quaternary ammonium compound.

19. The method of using a disinfecting device as recited in claim 18, wherein the disinfecting solution further comprises at least one drying element selected from a group consisting of a calcium sulfate, a sodium sulfate, a calcium chloride and a magnesium sulfate, and further wherein the at least one drying element is between 0.5 and 15% of the disinfecting solution by weight.

20. The method of using a disinfecting device as recited in claim 16, wherein the step of dispersing occurs at a flow rate of up to 0.52 quarts per minute.
